(19) United States
(12) Patent Application Publication
(10) Pub. No.: US 2021/0313023 A1
(43) Pub. Date: Oct. 7, 2021

(54) HELP NOW SYSTEM

(71) Applicant: Steven Hill, Dublin, OH (US)

(72) Inventor: Philip Payne, Dublin, OH (US)

(73) Assignee: Steven Hill, Dublin, OH (US)

(21) Appl. No.: 17/222,813

(22) Filed: Apr. 5, 2021

Related U.S. Application Data
(60) Provisional application No. 63/005,542, filed on Apr. 6, 2020.

(51) Int. Cl. G16H 10/60; G16H 40/67

(52) U.S. Cl. CPC G16H 10/60; G16H 40/67

(57) ABSTRACT

The subject technology is in the technical field of immediate crisis management for patients who are under physical, mental, or emotional distress, and who are in remote or difficult-to-access-locations. In particular, the subject technology serves such patients who recognize during “moments of clarity” that they need and want immediate help. Yet, they may be reluctant to seek help under normal circumstances because of a need to conceal the problem from others. The subject technology is a system and methods using cellular, Internet, geo-location, and other technology that make assistance available upon a simple action (clicking an icon), and deliverable through mobile or desktop devices. The system accommodates patients who remain anonymous, with information continuity across anonymous session, and those who choose to identify themselves in order to seek more extensive assistance.

### CROSS-REFERENCE TO RELATED APPLICATIONS

This application claims the benefit of U.S. Provisional Application No. 63/005,542, filed Apr. 6, 2020.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE TECHNOLOGY

The subject technology is in the technical field of immediate crisis management for patients who are under physical, mental, or emotional distress, and who are in remote or diff cult-to-access-locations. In particular, the subject technology serves such patients who recognize during “moments of clarity” that they need and want help. Yet, they may be reluctant to seek help under normal circumstances because of a need to conceal the problem from others.

### SUMMARY OF THE TECHNOLOGY

People with drug and others under physical, mental, or emotional distress, who are often in remote or difficult-to-access-locations, nay recognize during “moments of clarity” that they need and want help. Yet, they may be reluctant to seek help under normal circumstances because of a need to conceal the problem from others: loved ones, abusers, employers, government agencies, or others. Today, the cellular telephone and the use of smart telephones allows people to seek help when in remote and secluded locations. Also, of course, the Internet provides such access to patients without smart telephones, but have access to the Internet. These are aspects of the emerging telehealth and telemedicine industry.

What is needed is a way for the person, who is considered to be a “patient” even if anonymous, in such a remote or secluded location to take at least initial steps requesting assistance without revealing the request to certain others, and also providing an easy path for more substantial immediate and future help.

The subject technology is a system and methods using cellular, Internet, geo-location, and other technology that make assistance available upon a simple action (clicking an icon), and deliverable through mobile or desktop devices. The system offers two layers or modes of assistance:

1) free, immediate and anonymous counseling assistance for patients who recognize that they need help; counselors would be local when possible, but the system will be staffed for coverage regardless of location;

and

2) more extensive and billable treatment after the patient who chooses or commits to getting longer-term assistance; providers would be local when possible, but the system will be staffed for coverage regardless of location; care will be deliverable through telemedicine and physical presence, as required or available under the circumstance.

While the patient is still anonymous, certain limited information is retained so that if the anonymous patient reconnects later and still wants to remain anonymous, the system can provide continuity in treatment.

A solution is the Help Now System, as further described below.

### BRIEF DESCRIPTION OF THE DRAWINGS

FIG. 1 shows a topology and connections among patients, clinicians, and the Help Now System via a cloud combination of Internet and cellular networks.

FIG. 2 shows basic components of the Help Now System.

FIG. 3A shows a basic exchange through which clinicians state their general availability to receive requests for assistance.

FIG. 3B shows a sequence of inquiry in which the Help Now System receives a request for assistance from a patient, selects and offers specific contact opportunities, and makes the connection between patient and clinician who accepts to offer.

FIG. 4 shows communication between patient and clinician, and the cooperation of internal components of the Help Now System to provide and maintain the communication.

### DETAILED DESCRIPTION OF THE TECHNOLOGY

In the following description, reference may be made to elements not shown in the particular figure, but shown and discussed with respect to other figures.

The table below discloses reference numbers used in this specification and drawings for this application.
100 Help Now System
102 clinician database subsystem
104 clinician service
105 link
106 clinician database
107 link
108 notification subsystem
109 link
110 notification service
112 video subsystem
114 video service
116 video platform
200 cloud
202 site
203 communication
204 patients
206 browser
208 site
209 communication
210 clinicians
212 browser
220 connection
300 check-in sequence
302 notification sequence
304 clinician sequence
306 activation
308 notice
310 acknowledgment
312 activation
330 engagement sequence
332 patient sequence
334 notification service sequence
336 declining clinician sequence
338 accepting clinician sequence
340 request
341 associated activity
342 activation
344 activation
346 offer
348 activation
350 refusal
354 activation
358 activation
360 acceptance
370 activation
372 acknowledgment
374 acknowledgment
376 activation
380 video connection session
382 activity
404 communication
406 communication
410 setup
412 video connection
414 setup
416 video connection

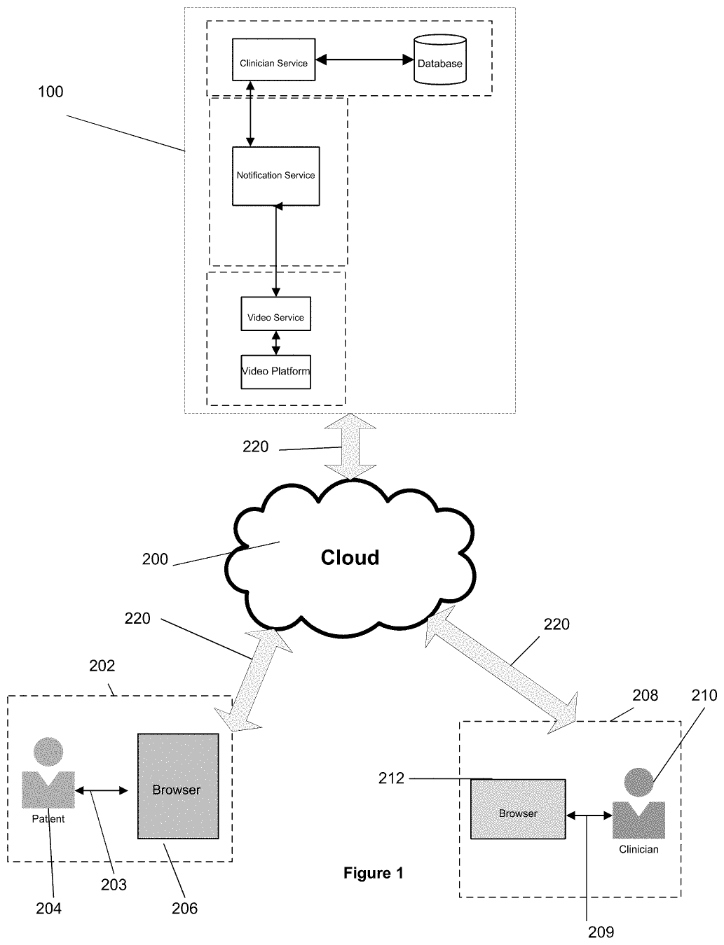

Referring now to FIG. 1, we show the basic topology showing among patients 204, clinicians 210, and the Help Now System 100 via a cloud 200 comprising a combination of Internet and cellular networks. The patient is at a site 202 and uses communication 203 to a browser 206 which is operating on a smart telephone connected to a cellular network or to the Internet, or other mobile device connected to the Internet. Similarly, the clinician 210 is at a site 208 and uses communication 209 to a browser 212 which is operating on a smart telephone connected to a cellular network or to the Internet, or other mobile device connected to the Internet, or desktop computer system connected to the Internet. The Help Now System 100 receives request from patient 204, finds a capable and available clinician 210, and completes an audio/video connection through the cloud 200 and connections 220.

The Help Now System 100 is currently designed as a web-based “single-page application,” wherein it interacts with users by dynamically rewriting a current web page with new data from an appropriate web server, as opposed to having the browser load entire new pages. The topology including the cloud 200 and the Help Now System 100 contemplates a variety of current and future implementation technologies. These include Google Cloud Platform, Microsoft Azure Cloud Computing Services, Amazon Web Services (“AWS”), and others. The current implementation of the Help Now System 100 employs Microsoft ASP. Net Core web application services.

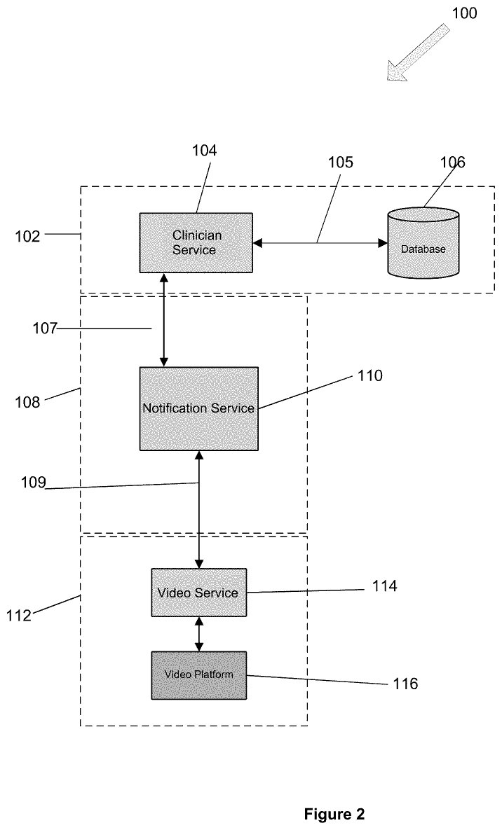

FIG. 2 show basic components of the Help Now System 100, comprising a Clinician Data Base Subsystem 102, a notification subsystem 108, and a video subsystem 112. The clinician database subsystem 102, in turn, further comprises a clinician service 104 and a clinician database 106. The video subsystem 112 further comprises a video service 114 and a video platform 116. The notification subsystem 108 further comprises notification service 110 and an appropriate application programming interface (“API”) from communication with the clinician service 104 and the video subsystem 112.

The clinician service 104 manages the flow of information from the clinician database 106 via link 105 and between the clinician database subsystem 102 and the notification subsystem 108 via link 107, by way of an appropriate API for that purpose. The clinician database 106 holds information about clinicians, as necessary for their possible selection based on patient needs. The information includes, but is not limited to, expertise, location, and general availability (as is discussed with respect to FIG. 2). The information further includes whether the clinician is currently engaged and thus unavailable for immediate services, and information about patients who have been or are being served.

The notification subsystem 108 is, in effect, the “heart” of the help Now System 100. It engages, as required, the clinician database subsystem 102 and the video subsystem 112 based on incoming requests for assistance and availability of clinicians. The notification subsystem 108 receives requests for assistance, searches for an appropriate and available clinician through the clinician database subsystem 102, offers the request to selected clinicians in turn until one accepts, and completes the communication between patient and clinician via the video subsystem 112 and link 109. The video platform 116 is a service providing video connection.

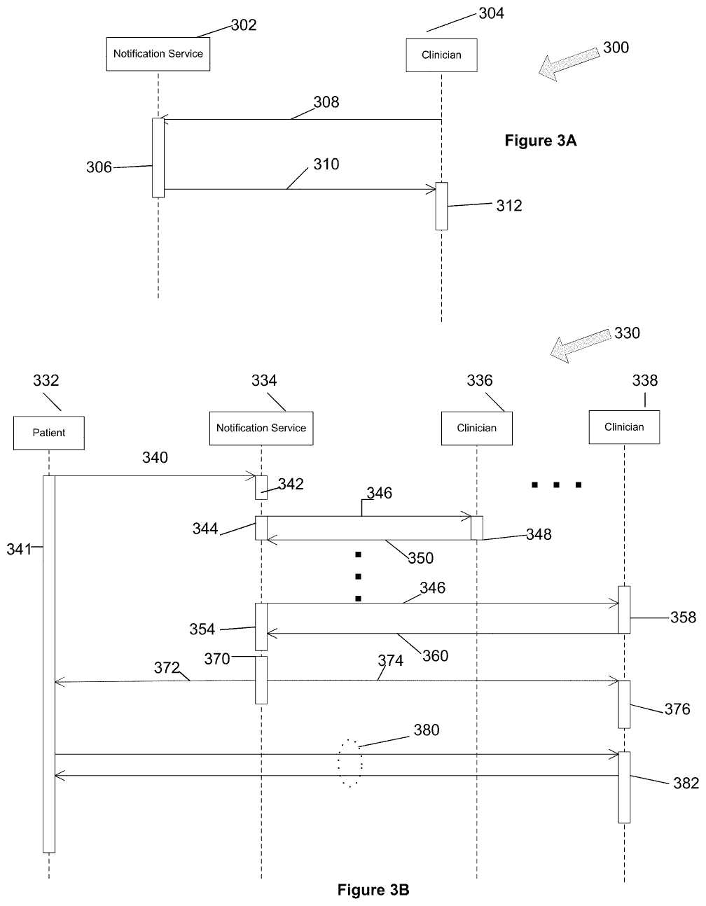

FIG. 3A shows a check-in sequence 300, comprising a basic exchange through which clinicians 210 state their general availability to receive requests for assistance. In a clinician sequence 304 initiated at the clinician site 208, by clinician 210, and operating through a browser 212, each clinician 210 indicates availability via a notice 308 via a check-in sequence 302 operating within the Help Now System 100 and the notification subsystem 108. Activation 306 updates an entry in the for the particular clinician through the clinician database subsystem 102 and gives an acknowledgement 310 to the clinician. The clinician 210 then begins a wait activation 312, performing other duties until a request to provide assistance arrives.

FIG. 3B shows an engagement sequence 330, comprising a patient sequence 332, a notification service sequence 334, a declining clinician sequence 336, and an accepting clinician sequence 338. Patient sequences are initiated by the patient 210 seeking help. From patient sequence 332 and associated activity 341, a request 340 is presented to the notification sequence 334. Within the notification subsystem 108 and queries to the clinician database subsystem 102, a prospective clinician 210 who meets location and other criteria is selected. Via activity 344, an offer 346 is presented to the prospective clinician 210. Declining clinician sequence 336 shows what happens when the prospective clinician 210 declines the offer 346, or does not accept within a certain time period. In such case, activation 348 returns a refusal 350 to the notification service sequence 334 via activation 344. The notification service sequence 344 proceeds though other prospective clinicians 210 until a clinician 210 accepts via an accepting clinician sequence 338. In the accepting clinician sequence 338, request 346 arrives. Via activation 358, and acceptance 360 is returned to activation 354, which in turn causes activation 370 to begin. Activation 370 initiates acknowledgement 372 and 374, further causes a video connection session 380 to arise via activity 382 between patient and accepting clinician. The session 380 continues until terminated by either the patient 204 or clinician 210.

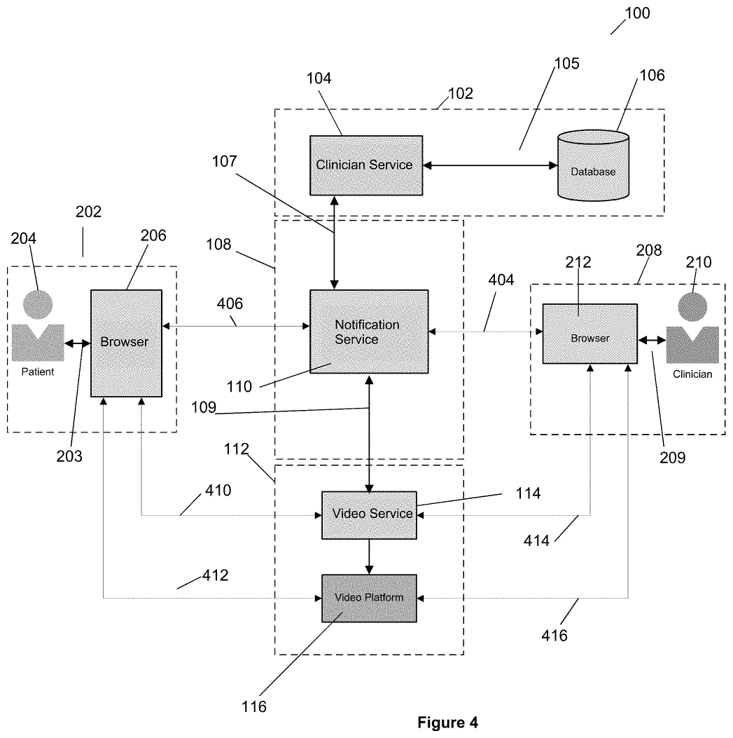

FIG. 4 is another view of communication sequences between patient 204 and clinician 210, and the cooperation of internal components of the Help Now System 100. The description above of FIG. 2 applies here as well, with the addition of
the patient 204 at patient site 202 with communication 203 through browser 206
the clinician 210 at clinician site 208 with communication 208 through browser 212.

The following additions are further representations of connections 220 and the cloud 200:
communication 406 between notification service 110 and patient browser 206
communication 404 between notification service 110 and clinician browser 212
setup 410 between video service 114 and patient browser 206
setup 414 between video service 114 and clinician browser 212
video connection 412, extending through video platform 116 and video connection 416, to clinician browser 414.

Embedded within the Help Now System 100 is “role based access” to information and services. Contemplated roles include patients 204 and clinicians 210, as well as various administrative roles, including system administrator, organization administrator, clinical administrator, and others who may be allowed certain access to case information. Only those in appropriate roles have access to portions of information, as determined by law, policy, and certainly patient permission.

Single Action Request

Referring still to FIGS. 4 and 3B, the Help Now System 100 presents to the patient 204, through the browser 206 presented via smart telephone, other mobile device, or desktop system, an icon or similar user interface mechanism that the patient 204 need only to press or otherwise select to initiate the request 340.

Free, Anonymous Mode

Still referring to FIG. 4, the Help Now System 100 provides a free, immediate and anonymous mode of counseling assistance for a patient 104 who recognizes a need for help but does not want to reveal identity, Patient 104 may seek help a later time while remaining anonymous. It is beneficial for the patient 104 for the clinician 210 to have access to information from previous requests. The Help Now System 100 provides that feature. Key to anonymous mode processing is that on initial request 340 by a patient 104, the Help Now System 100, through the notification service 100, records tracking information about the device used by the patient 104, such as telephone number, Media Access Control (“MAC”) address, and geographical location of site 202 via latitude and longitude if available. Although not entirely definitive, this tracking information provides some association with the patient 104 who may wish to remain anonymous. Tracking information may include other information volunteered by the patient. Location of site 202 may change, the patient 104 may use different devices. Furthermore, through the use of Internet cookies or tokens or similar mechanisms, the Help Now System 100 may deliver tagging information to the patient 104 or the device, to be remembered by the patient 104 or stored in the device. This tagging information as a further step in associating a patient who wants to remain anonymous, to a previous session. And yet no presumption of association is made. Statistical analysis within the Help Now System 100 provides a confidence level of association, and will be used by the clinician to use holistic means and experience to interview the patient to confirm any prior association. Precision, accuracy, and delicacy is required in order not to upset or alarm the patient 104.

Billable, Commitment Mode

The Help Now System 100 provides more extensive and billable treatment after the patient 104 who chooses or commits to getting longer-term assistance, and thus is no longer anonymous. The patient 104 must volunteer identifying information when requesting extensive treatment. Such information is retained in the Help Now System 100, and used by the clinician 210 to recommend other clinicians or providers as may be appropriate, for treatment delivered online or in-person. Furthermore, extensive tagging information is provided to the patient 104 by the Help Now System 100 in compliance with all legal and ethical requirements to safeguard information and to be certain t clinician 210 is treating the particular patient 104.

Variations and Additions

We anticipate that the system will include other features, including but not limited to:
refining the implementation of anonymous mode accounting for use by a patient using non-private facilities such as at a library
selecting clinicians based on rating systems as well as by location

While the foregoing written description enables one of ordinary skill to make and use what is considered presently to be the best mode thereof, those of ordinary skill will understand and appreciate the existence of variations, combinations, and equivalents of the specific embodiment, method, and examples herein. The Help Now System described here should therefore not be limited by the above described embodiments, methods, underlying web services technologies, or examples, but by all embodiments and methods within the scope and spirit of the subject technology.

1. A help now system providing a communication session between a patent and a clinician comprising,
a clinician database subsystem;
a notification subsystem;
a video subsystem;
said clinician database subsystem, said notification subsystem, and video subsystem communicating across a cloud;
wherein the help now system accommodating the patient who remains anonymous;
wherein the help now system accommodating the patient who volunteers identifying information;
wherein the help now system retains tracking information to assist in associating the patient who remains anonymous with subsequent sessions;
wherein the help now system controls access to patient information based on role of the requestor; and
wherein the help now system performs statistical analysis to provide a confidence level of accurate association between the patient and a previous session.

2. The help now system of claim 1 wherein the clinician is selected upon criteria comprising location of the patient;
location of the one or more clinicians;
nature of need;
expertise of the clinician; and
rating of the clinician.

3. A method of providing continuing services to patients from one or more clinicians through a help now system comprising the steps of
the patient makes anonymous request for services through the help now system;
the patient provides minimal information to support of future requests for services;
the help now system derives information to preserve association in support of future requests for services, the derived information maintaining anonymity for the patient;
the help now system assigns the one or more clinicians to provide services to the patient;
the one or more clinicians provide services as are appropriate;
the patient receives services from the one or more clinicians according to available information, policy, and law; and
if the patient removes anonymity, then the one or more clinicians provide additional services and information as are appropriate.
